(19) United States
(12) Patent Application Publication
(10) Pub. No.: US 2022/0117916 A1
(43) Pub. Date: Apr. 21, 2022

(54) Combination Products to Mitigate the Risk of Non-Benzodiazepine Benzodiazepine Agonist Adverse Reaction and Overdose

(71) Applicant: SafeRx Pharmaceuticals, LLC, Lenox, MA (US)

(72) Inventor: Michael Presti, Lenox, MA (US)

(21) Appl. No.: 17/238,459

(22) Filed: Apr. 23, 2021

Related U.S. Application Data
(60) Provisional application No. 63/093,714, filed on Oct. 19, 2020.

(51) Int. Cl. A61K 31/145; A61K 31/275; A61K 31/04; A61K 31/7048; A61K 31/4164; A61K 31/198; A61K 31/55; A61K 31/137; A61K 31/4184; A61K 31/11; A61K 31/437; A61K 31/4985; A61K 31/519; A61K 31/4375; A61K 31/496; A61K 31/438; A61K 31/4709; A61K 31/5025; A61K 31/4439; A61P 25/32

(52) U.S. Cl. CPC A61K 31/145; A61P 25/32; A61K 31/04; A61K 31/7048; A61K 31/4164; A61K 31/198; A61K 31/55; A61K 31/137; A61K 31/4184; A61K 31/11; A61K 31/437; A61K 31/4985; A61K 31/519; A61K 31/4375; A61K 31/496; A61K 31/438; A61K 31/4709; A61K 31/5025; A61K 31/4439; A61K 31/275

(57) ABSTRACT

A method is provided for reducing a risk that a subject will experience a medically-related adverse event associated with inappropriate consumption of alcohol concurrent with the taking of a nonbenzodiazepine benzodiazepine receptor agonist (NBBRA). In an example, the method comprises administering to the subject a combination product that includes an effective amount of one or more NBBRAs, and an effective amount of one or more aldehyde dehydrogenase inhibitors (ALDIs), to provide the desired effects of the NBBRA in conjunction with a substance that prevents or deters concurrent alcohol consumption, thereby reducing the risk of the subject experiencing an alcohol-induced medically-related adverse event. Also disclosed are the combination products that include the effective amount of one or more NBBRAs and the effective amount of one or more ALDIs.

### CROSS-REFERENCE TO RELATED APPLICATIONS

This application claims the benefit under 35 USC § 119(e) of U.S. Provisional Application No. 63/093,714, filed Oct. 19, 2020, which is incorporated herein by reference in its entirety for all purposes.

### FIELD OF THE DISCLOSURE

The present disclosure pertains to combination drug products and methods of use thereof for reducing a risk of medication-related adverse effects including injury, undesirable behaviors, and death, associated with consumption of alcohol in conjunction with nonbenzodiazepine benzodiazepine receptor agonists.

### BACKGROUND

Nonbenzodiazepine benzodiazepine receptor agonists (NBBRAs) represent a subtype of commonly prescribed sedative-hypnotic sleep aids. Examples include but are not limited to zolpidem (Ambien™), eszopiclone (Lunesta™), and zaleplon (Sonata™). NBBRAs are often favored over benzodiazepine sleep aids because of their enhanced safety profile. However, NBBRAs still pose a risk of serious medication-related adverse effects, including but not limited to injury and/or death. The risk of such adverse events is influenced by numerous factors such as patient age, comorbidities, dose of medication, and duration of use, as well as biochemical interactions with other ingested substances.

Of the above factors that influence the risk of adverse events associated with use of NBBRAs, biochemical interactions with other ingested substances is one of the biggest contributors. For example, the likelihood of a potentially severe NBBRA-related adverse event (e.g., death) can be significantly increased when these medications are combined with other centrally acting substances that enhance inhibitory GABA-ergic neurotransmission, also known as central nervous system (CNS) depressants. For example, concurrent consumption of alcohol with NBBRAs can increase a risk of medication-related overdose requiring intensive care unit (ICU) admission. As other examples, concurrent consumption of alcohol with NBBRAs can increase a risk of complex sleep-related behaviors such as parasomnia (e.g., sleep walking, sleep eating, sleep driving, etc.) and amnestic behavior, which can contribute to injury and/or death. Although patients are routinely advised to avoid consumption of CNS depressants in conjunction with NBBRAs, large portions of patients continue to use CNS depressant(s) (e.g., alcohol) on a regular basis while taking NBBRAs.

### SUMMARY

Disclosed is a method of reducing the chances of a subject experiencing a medically-related adverse event stemming from concurrent ingestion of alcohol whilst taking an NBBRA. In embodiments, the method includes administering a combination drug product (e.g., combination medication) to the subject, the combination product including at least an effective amount of one or more NBBRAs and an effective amount of one or more aldehyde dehydrogenase inhibitors (ALDIs). The combination product may be in the form of a pill or other formulation (e.g., capsule, sublingual film, oral spray) that resists (e.g., makes extremely challenging) or entirely prevents separation of the one or more NBBRAs from the one or more ALDIs. The combination product may thus provide the desired effects (e.g., sedation, reduced anxiety, and the like) of the NBBRA(s) while reducing the risk of medically-related adverse effects caused by the concurrent ingestion of alcohol.

Also disclosed is a combination medication that includes an effective amount of one or more NBBRAs, an effective amount of one or more ALDIs, and optionally, a pharmaceutically acceptable carrier.

In embodiments, the ALDI includes one or more of disulfiram, calcium carbimide, coprine, cyanamide, 1-aminocyclopropanol, daidzin, cephalosporins, antidiabetic sulfonyl ureas, metronidazole, ampal, benomyl, citral and active isomers thereof, chloral hydrate, chlorpropamide analogs NPI-1 and API-1, CVT-10216, DEAB, gossypol, kynurenine tryptophan metabolites, molinate, nitroglycerin, pargyline, and active metabolites, analogs, or pharmaceutically acceptable salts thereof. In specific examples, the ALDI comprises, consists essentially of, or consists of disulfiram, active disulfiram metabolites and/or pharmaceutically acceptable salts thereof. In embodiments, the ALDI consists essentially of disulfiram, active metabolites and/or pharmaceutically acceptable salts thereof.

In embodiments, the one or more NBBRAs include one or more of zolpidem, zaleplon, pagoclone, gedocarnil, indiplon, eszopiclone, zopiclone, ocinaplon, alpidem, saripidem, necopidem, suproclone, pazinaclone, suriclone, divaplon, fasiplon, lorediplon, taniplon, panadiplon, abecarnil, ZK-93423, gedocarnil, SL-651,498, CGS-20625, Y-23684, CGS-9896, TPA-023, CL-218,872, NS-2664, TP-003, NS-2710, TP-13, SX-3228, L-838,417, NS-2710, ELB-139, RWJ-51204, GBLD-345, pipequaline, SB-205,384, SX-3228, mixtures of any of the foregoing, salts of any of the foregoing, and the like.

In one aspect, the present disclosure provides a method of reducing the chances of an alcohol-mediated nonbenzodiazepine benzodiazepine receptor agonist (NBBRA)-related adverse event occurring during NBBRA usage, comprising administering a single composition to a subject in need of a NBBRA, the single composition comprising an effective amount of one or more NBBRAs for the management of a sleep or anxiety disorder; and an effective amount of one or more aldehyde dehydrogenase inhibitors (ALDIs) sufficient to prevent alcohol consumption. The one or more ALDIs may be selected from disulfiram, calcium carbimide, coprine, cyanamide, 1-aminocyclopropanol, daidzin, cephalosporins, antidiabetic sulfonyl ureas, metronidazole, ampal, benomyl, citral and active isomers thereof, chloral hydrate, chlorpropamide analogs, (benzoyloxy)[4-chlorophenyl)sulfonyl]carbamic acid 1,1 dimethyl ester (NPI-1), 4-chloro-N-ethyl-N-[(propylamino)carbonyl]benzenesulfonamid (API-1), 3-(((3-(4-(methylsulfonamido)phenyl)-4-oxo-4H-chromen-7-yl)oxy)methyl) benzoic acid (CVT-10216), N,N-diethylaminobenzaldehyde (DEAB), gossypol, molinate, nitroglycerin, pargyline, or pharmaceutically acceptable salts thereof, enabling provision of the effects of the one or more NBBRAs in a manner which prevents concomitant alcohol consumption, thereby reducing the risk of alcohol-mediated NBBRA-related adverse events during NBBRA usage for management of sleep and/or anxiety disorders.

In some embodiments, the ALDI is disulfiram, or a pharmaceutically acceptable salt thereof.

In some embodiments, the one or more NBBRAs is one or more prescription NBBRAs.

In some embodiments, the one or more NBBRAs comprises one or more of zolpidem, zaleplon, pagoclone, gedocarnil, indiplon, eszopiclone, zopiclone, ocinaplon, alpidem, saripidem, necopidem, suproclone, pazinaclone, suriclone, divaplon, fasiplon, lorediplon, taniplon, panadiplon, abecarnil, ZK-93423, gedocarnil, SL-651,498, CGS-20625, Y-23684, CGS-9896, TPA-023, CL-218,872, NS-2664, TP-003, NS-2710, TP-13, SX-3228, L-838,417, NS-2710, ELB-139, RWJ-51204, GBLD-345, pipequaline, SB-205,384, and SX-3228. For example, in an embodiment the one or more NBBRAs is zolpidem, eszopiclone, or zaleplon. In some embodiments, the one or more NBBRAs consists essentially of zolpidem, eszopiclone, or zaleplon.

In some embodiments, the subject is prescribed one or more NBBRAs for the management of the sleep disorder.

In some embodiments, the subject is prescribed one or more NBBRAs for the management of the anxiety disorder.

In one aspect, the present disclosure provides a method of reducing the chances of an alcohol-mediated nonbenzodiazepine benzodiazepine receptor agonist (NBBRA)-related adverse event occurring during NBBRA usage, comprises administering to a subject in need of a NBBRA a single combination medication, comprising an effective amount of an NBBRA for the management of a sleep and/or an anxiety disorder selected from zolpidem, zaleplon, pagoclone, gedocarnil, indiplon, eszopiclone, zopiclone, ocinaplon, alpidem, saripidem, necopidem, suproclone, pazinaclone, suriclone, divaplon, fasiplon, lorediplon, taniplon, panadiplon, abecarnil, ZK-93423, gedocarnil, SL-651,498, CGS-20625, Y-23684, CGS-9896, TPA-023, CL-218,872, NS-2664, TP-003, NS-2710, TP-13, SX-3228, L-838,417, NS-2710, ELB-139, RWJ-51204, GBLD-345, pipequaline, SB-205,384, or SX-3228; and an effective amount of one or more aldehyde dehydrogenase inhibitors (ALDIs) selected from disulfiram, calcium carbimide, coprine, cyanamide, 1-aminocyclopropanol, daidzin, cephalosporins, antidiabetic sulfonyl ureas, metronidazole, ampal, benomyl, citral and active isomers thereof, chloral hydrate, chlorpropamide analogs, (benzoyloxy)[4-chlorophenyl)sulfonyl]carbamic acid 1,1 dimethyl ester (NPI-1), 4-chloro-N-ethyl-N-[(propylamino)carbonyl]benzenesulfonamid (API-1), 3-(((3-(4-(methylsulfonamido)phenyl)-4-oxo-4H-chromen-7-yl)oxy)methyl) benzoic acid (CVT-10216), N,N-diethylaminobenzaldehyde (DEAB), gossypol, molinate, nitroglycerin, pargyline, or pharmaceutically acceptable salts thereof, enabling provision of the effects of the one or more NBBRAs in a manner which prevents concomitant alcohol consumption, thereby reducing the risk of alcohol-mediated NBBRA-related adverse events during NBBRA usage for management of sleep and/or anxiety disorders.

In some embodiments, the ALDI is disulfiram, or a pharmaceutically acceptable salt thereof. In some embodiments the ALDI consists essentially of disulfiram, or a pharmaceutically acceptable salt thereof.

In some embodiments, the one or more NBBRAs comprises zolpidem, eszopiclone, or zaleplon. In some embodiments, the one or more NBBRAs consists essentially of one or more of zolpidem, eszopiclone, or zaleplon.

In some embodiments, the subject is prescribed one or more NBBRAs for the management of the sleep disorder.

In some embodiments, the subject is prescribed one or more NBBRAs for the management of the anxiety disorder.

In one aspect, the present disclosure provides a combination medication, comprising an effective amount of one or more nonbenzodiazepine benzodiazepine receptor agonists (NBBRAs), and an effective amount of one or more aldehyde dehydrogenase inhibitors (ALDIs) and a pharmaceutically acceptable carrier, wherein the one or more ALDIs is selected from one or more of disulfiram, calcium carbimide, coprine, cyanamide, 1-aminocyclopropanol, daidzin, cephalosporins, antidiabetic sulfonyl ureas, metronidazole, ampal, benomyl, citral and active isomers thereof, chloral hydrate, chlorpropamide analogs (benzoyloxy)[4-chlorophenyl)sulfonyl]carbamic acid 1,1-dimethylethyl ester (NPI-1) and 4-chloro-N-ethyl-N-[(propylamino)carbonyl]benzenesulfonamid (API-1), 3-(((3-(4-(methylsulfonamido)phenyl)-4-oxo-4H-chromen-7-yl)oxy)methyl)benzoic acid (CVT-10216), N,N-diethylaminobenzaldehyde (DEAB), gossypol, kynurenine tryptophan metabolites, molinate, nitroglycerin, pargyline, S-methyl N,N-diethyldithiocarbamate, S-methyl N,N-diethyldithiocarbamate sulfoxide, and S-methyl N,N-diethylthiocarbamate sulfoxide, HNO, 1-aminocyclopropanol (ACP), thioampal, 2-mercaptobenzothiazole (MBT), molinate sulfoxide, molinate sulfone, NO3, propiolaldehyde, or pharmaceutically acceptable salts thereof.

In some embodiments, the ALDI is disulfiram, or a pharmaceutically acceptable salt thereof. In some embodiments, the ALDI consists essentially of disulfiram, or a pharmaceutically acceptable salt thereof.

In some embodiments, the one or more NBBRAs is one or more prescription NBBRAs.

In some embodiments, the one or more NBBRAs is one or more of zolpidem, zaleplon, pagoclone, gedocarnil, indiplon, eszopiclone, zopiclone, ocinaplon, alpidem, saripidem, necopidem, suproclone, pazinaclone, suriclone, divaplon, fasiplon, lorediplon, taniplon, panadiplon, abecarnil, ZK-93423, gedocarnil, SL-651,498, CGS-20625, Y-23684, CGS-9896, TPA-023, CL-218,872, NS-2664, TP-003, NS-2710, TP-13, SX-3228, L-838,417, NS-2710, ELB-139, RWJ-51204, GBLD-345, pipequaline, SB-205,384, and SX-3228. In some embodiments, the one or more NBBRAs is zolpidem, eszopiclone, or zaleplon. In some embodiments, the one or more NBBRAs consists essentially of one or more of zolpidem, eszopiclone, or zaleplon.

### DETAILED DESCRIPTION

Unless otherwise explained, all technical and scientific terms used herein have the same meaning as commonly understood by one of ordinary skill in the art to which the disclosed subject matter belongs. Definitions of common terms in chemistry terms may be found in The McGraw-Hill Dictionary of Chemical Terms, 1985, and The Condensed Chemical Dictionary, 1981.

As used herein, the singular terms “a,” “an,” and “the” include plural referents unless context clearly indicates otherwise. Similarly, the word “or” is intended to include “and” unless the context clearly indicates otherwise. Also, as used herein, the term “comprises” means “includes.” Hence “comprising A or B” means including A, B, or A and B. Except as otherwise noted, any quantitative values are approximate whether the word “about” or “approximately” or the like are stated or not. The materials, methods, and examples described herein are illustrative only and not intended to be limiting. Any molecular weight or molecular mass values are approximate and are provided only for description.

Except as otherwise noted, the methods and techniques of the present disclosure are generally performed according to conventional methods well known in the art and as described in various general and more specific references that are cited and discussed throughout the present specification. See, e.g., Loudon, Organic Chemistry, Fourth Edition, New York: Oxford University Press, 2002, pp. 360-361, 1084-1085; Smith and March, March's Advanced Organic Chemistry: Reactions, Mechanisms, and Structure, Fifth Edition, Wiley-Interscience, 2001; or Vogel, A Textbook of Practical Organic Chemistry, Including Qualitative Organic Analysis, Fourth Edition, New York: Longman, 1978.

In case of conflict, the present specification, including explanations of terms, will control.

To facilitate review of the various embodiments of this disclosure, the following explanations of specific terms are provided:

Administration: To provide or give a subject a composition, such as a pharmaceutical composition including an ALDI and an NBBRA medication formulated as a combination drug product, by any effective route. Exemplary routes of administration include, but are not limited to, injection (such as subcutaneous, intramuscular, intradermal, intraperitoneal (ip), and intravenous (iv)), oral, sublingual, transdermal, and inhalation routes.

Aldehyde dehydrogenases: Enzymes of enzyme class (EC) 1.2.1.3 that catalyze the oxidation of aldehyde. Aldehyde dehydrogenases comprise the primary enzymes involved in alcohol metabolism.

Aldehyde dehydrogenase inhibitor (ALDI): An inhibitor of the enzymatic activity of an aldehyde dehydrogenase. Examples of aldehyde dehydrogenase inhibitors include: disulfiram ([1-diethylthiocarbamoyldisulfanyl-N,N-diethylmethanethioamide]) and active metabolites thereof, such as S-methyl N,N-diethyldithiocarbamate, S-methyl N,N-diethyldithiocarbamate sulfoxide, and S-methyl N,N-diethylthiocarbamate sulfoxide; calcium carbimide, sold as the citrate salt under the trade name Temposil®; coprine and active metabolites thereof, such as 1-amino cyclopropanol; cyanamide and active metabolites thereof, such as HNO; 1-aminocyclopropanol and active metabolites thereof, such as ACP; daidzin; cephalosporins; antidiabetic sulfonyl ureas; metronidazole; ampal and active metabolites thereof, such as thioampal; benomyl (methyl [1-[(butylamino)carbonyl]-1H-benzimidazol-2-yl]carbamate) and active metabolites thereof, such as MBT; citral and active isomers thereof, such as neral and geranial; chloral hydrate; chlorpropamide analogs NPI-1 and API-1, CVT-10216, DEAB; gossypol, kynurenine tryptophan metabolites KA, 3-HK, and 3-HAA; molinate and active metabolites thereof, such as molinate sulfoxide and molinate sulfone; nitroglycerin and active metabolites thereof, such as NO₃; pargyline and active metabolites thereof, such as propiolaldehyde; and any other metabolites or analogs exhibiting aldehyde dehydrogenase inhibiting activity.

Combination product(s): As discussed herein pertain to pharmaceutical preparations that include at least a first agent and a second agent prepared in such a way as to prevent or resist separation of the active pharmaceutical agents into their individual counterparts. In examples, the first agent and the second agent may be included in a single tablet or other oral or sublingual formulation (e.g., tamper-resistant matrix formulation, oral spray, and the like) thereby preventing or resisting separation of the individual active pharmaceutical agents. As discussed herein, a combination product may be referred to as a combination drug, combination drug product, combination medication, or fixed-dose combination (FDC).

Contacting: Placement in direct physical association including both in solid or liquid form. Contacting can occur in vivo, for example by administering an agent (e.g., combination drug product) to a subject.

Effective amount: An amount of therapeutic agent that is sufficient to generate a desired response, such as reducing or inhibiting one or more signs or symptoms associated with a condition/disorder or disease. When administered to a subject, a dosage will generally be used that will achieve target tissue/cell concentrations. In some examples, an “effective amount” is one that prevents or reduces adverse reactions in response to consumption of a substance (e.g., CNS depressant such as alcohol) concomitantly with consumption of an NBBRA.

In particular examples, it is an amount of an agent capable of inhibiting one or more aldehyde dehydrogenase(s) from catalyzing the oxidation of acetaldehyde, for example following consumption of alcohol. In some examples, it is an amount of an agent that prevents or reduces a subject's desire for and/or consumption of an agent (e.g., CNS depressant such as alcohol) that, when combined with an NBBRA, can increase a risk of a medically-related adverse effect.

In examples, signs and/or symptoms of a medically-related adverse effect do not have to be completely inhibited for the pharmaceutical preparation to be effective. For example, a pharmaceutical preparation (e.g., an ALDI) may decrease signs or symptoms associated with concomitant consumption of an NBBRA and an agent that enhances inhibitory GABAergic neurotransmission (e.g., CNS depressant such as alcohol) by a desired amount, for example by at least 10%, at least 20%, at least 30%, at least 40%, at least 50%, at least 60%, at least 70%, at least 80%, at least 90% or even at least 100%, as compared to signs or symptoms in the absence of the pharmaceutical preparation. Such signs or symptoms may include but are not limited to parasomnias, injury (e.g., falls), death, and the like.

In examples, an effective amount is an amount of an NBBRA sufficient to elicit a desired effect, such as a desired degree of sedation and/or a desired degree of anti-anxiety effect.

Inhibiting or treating a disease or a condition: As discussed herein pertains to inhibiting the full development of a disease or condition, for example, in a subject who is in need of an NBBRA, such as a subject suffering from insomnia and/or anxiety. For example, treatment with an NBBRA may prevent the full development of insomnia in a subject.

In a particular example, the full development of a condition may comprise the full development of a medically-related adverse event (e.g., injury and/or death) resulting from concurrent ingestion of a substance that enhances inhibitory GABAergic neurotransmission (e.g., CNS depressant such as alcohol) and an NBBRA. In such an example, full development of the medically-related adverse event may be inhibited by combining an effective amount of an NBBRA with an effective amount of an ALDI in the form of a combination drug product, such that potential for full development of the medically-related adverse event may be reduced or avoided. “Treatment” refers to a therapeutic intervention that ameliorates a sign or symptom of a disease or pathological condition after it has begun to develop. For example, treatment with an NBBRA may ameliorate one or more signs or symptoms of insomnia. As another example, treatment with an ALDI may ameliorate one or more signs or symptoms of alcohol use or overconsumption (e.g., alcohol abuse). The term “ameliorating,” with reference to a disease or pathological condition, refers to any observable beneficial effect of the treatment. For example, an NBBRA may be used to ameliorate one or more signs or symptoms associated with sleep irregularities. As another example, an ALDI may be used to ameliorate one or more signs or symptoms associated with alcohol consumption (e.g., overconsumption, or any level of consumption that may increase a risk of a medically-related adverse event, organ damage (e.g., liver damage), and the like). The beneficial effect can be evidenced, for example, by a delayed onset of clinical symptoms of the disease/condition in a susceptible subject, a reduction in severity of some or all clinical symptoms of the disease, such as sleep disturbances, a slower progression of the disease/condition, an improvement in the overall health or well-being of the subject, or by other clinical or physiological parameters associated with a particular disease/condition. In examples, a combination drug product comprising one or more ALDIs and one or more NBBRAs may be used to ameliorate undesirable medically-related events (e.g., parasomnias, injury, death, etc.), while also ameliorating one or more signs or symptoms associated with sleep irregularities and/or anxiety disorder(s). A “prophylactic” treatment as discussed herein may pertain to a treatment administered to a subject who does not necessarily exhibit signs of a particular condition (e.g., medically-related adverse event) but who may be at risk of development of the particular condition (e.g., medically-related adverse event) if the treatment (e.g., combination of an ALDI with an NBBRA) is not provided. For example, a person with a history of alcohol abuse may be given an NBBRA medication as a combination drug product that also includes one or more ALDIs in order to reduce the chance of an undesirable medically-related event occurring while taking the NBBRA for the desired purpose.

Inhibit: To reduce to a measurable extent. For example, to reduce the incident or chance of alcohol-mediated or alcohol-exacerbated medically-related adverse events.

Medically-related adverse event: As discussed herein, pertains to an adverse event that is precipitated by combining a therapeutic agent with another agent that causes an interaction in the body of a subject leading to the adverse event. In examples, the therapeutic agent is an NBBRA, and the other agent is alcohol (or other CNS depressant). Medically-related adverse events as herein discussed comprise events that would either not occur or would occur with reduced detrimental consequences and/or severity to the subject in the absence of the other agent such as alcohol. Medically-related adverse events can include but are not limited to parasomnia (e.g., sleep walking, sleep eating, sleep driving), amnesia, overdose, death, injury, and the like. In examples disclosed herein, medically-related adverse events can be reduced or avoided by combining one therapeutic agent (e.g., NBBRA) with another therapeutic agent (e.g., ALDI), for example in the form of a combination product. Discussed herein, medically-related adverse events may also be referred to as undesirable medically-related events, or NBBRA-related adverse events.

Nonbenzodiazapines or nonbenzodiazepine benzodiazepine receptor agonist (NBBRA): Drugs used for treatment of conditions including but not limited to insomnia and other sleep disorders, anxiety disorders, panic disorders, and the like. Nonbenzodiazepine pharmacodynamics are similar to benzodiazepine drugs, however nonbenzodiazepines are unrelated to benzodiazepines on a molecular level (e.g., chemical structure). Nonbenzodiazepines are positive allosteric modulators of the GABA_{A} receptor, and they bind to and activate the site of the GABA_{A} receptor complex to which benzodiazepines bind. Nonbenzodiazepines enhance the effect of GABA by lowering a concentration of GABA required to open the GABA channel, thereby enhancing GABA's inhibitory effect. Nonbenzodiazepines are often prescribed preferably to benzodiazepines due to nonbenzodiazepines being perceived as more effective and safer, producing less tolerance, addiction, and dependence than benzodiazepines, although this perception is still debated. Nonbenzodiazepines are associated with safety risks, including but not limited to being associated with respiratory depression, CNS depression when taken with alcohol, unpredictable paradoxical reactions including but not limited to hyperactivity, increased hostility, increased aggression, anxiety, acute excitement, vivid dreams, sexual disinhibition, impaired driving performance, parasomnia, amnesia, and the like. Nonbenzodiazepines have the potential for one or more of abuse, tolerance, and withdrawal.

Examples of NBBRs include but are not limited to zolpidem, zaleplon, pagoclone, gedocarnil, indiplon, eszopiclone, zopiclone, ocinaplon, alpidem, saripidem, necopidem, suproclone, pazinaclone, suriclone, divaplon, fasiplon, lorediplon, taniplon, panadiplon, abecarnil, ZK-93423, gedocarnil, SL-651,498, CGS-20625, Y-23684, CGS-9896, TPA-023, CL-218,872, NS-2664, TP-003, NS-2710, TP-13, SX-3228, L-838,417, NS-2710, ELB-139, RWJ-51204, GBLD-345, pipequaline, SB-205,384, SX-3228, mixtures of any of the foregoing, salts of any of the foregoing, and the like.

Pharmaceutically acceptable carriers: The pharmaceutically acceptable carriers of use are conventional. Remington's Pharmaceutical Sciences, by E.W. Martin, Mack Publishing Co., Easton, Pa., 19th Edition, 1995, describes compositions and formulations suitable for pharmaceutical delivery of the compositions disclosed herein.

In general, the nature of the carrier will depend on the particular mode of administration being employed. For instance, parenteral formulations usually comprise injectable fluids that include pharmaceutically and physiologically acceptable fluids such as water, physiological saline, balanced salt solutions, aqueous dextrose, glycerol or the like as a vehicle. For solid compositions (such as powder, pill, tablet, or capsule forms), conventional non-toxic solid carriers can include, for example, pharmaceutical grades of mannitol, lactose, starch, or magnesium stearate. In addition to biologically neutral carriers, pharmaceutical compositions to be administered can contain minor amounts of non-toxic auxiliary substances, such as wetting or emulsifying agents, preservatives, and pH buffering agents and the like, for example sodium acetate or sorbitan monolaurate.

Subject: The term “subject” includes both human and veterinary subjects, for example, humans, non-human primates, dogs, cats, horses, rats, mice, and cows. Similarly, the term mammal includes both human and non-human mammals. In some embodiments, a subject is a patient, such as patient prescribed one or more NBBRA medications.

Therapeutic agent or Pharmaceutical agent: A chemical compound, small molecule, or other composition capable of inducing a desired therapeutic or prophylactic effect when properly administered to a subject, for example reducing the chances of an NBBRA-related adverse medical event.

Therapeutically effective amount or Effective amount: The amount of agent, such as an ALDI and an NBBRA medication, that is sufficient to prevent, treat (including prophylaxis), reduce and/or ameliorate the symptoms and/or underlying causes of any of a disorder, condition, or disease. For example, a combination drug product comprising an ALDI and an NBBRA may be used to treat, prevent, inhibit, and/or reduce chances of an NBBRA-related adverse event occurring due to the concomitant consumption of an NBBRA and alcohol. A therapeutically effective amount of an NBBRA may be an amount that induces a desired effect, such as a desired level of sedation and/or desired level of anxiety reduction, in a subject suffering from such conditions. A therapeutically effective amount of an ALDI may be an amount that substantially reduces or entirely prevents a subject from imbibing alcohol.

Suitable methods and materials for the practice or testing of this disclosure are described below. Such methods and materials are illustrative only and are not intended to be limiting. Other methods and materials similar or equivalent to those described herein can be used. For example, conventional methods well known in the art to which this disclosure pertains are described in various general and more specific references. In addition, the materials, methods, and examples are illustrative only and not intended to be limiting.

Overview

Nonbenzodiazepine medications are commonly prescribed to treat sleep problems, most commonly chronic insomnia, with potentially lower risk of side effects when compared to benzodiazepines. The mechanism of action for nonbenzodiazepines is similar to that of benzodiazepines. They are benzodiazepine receptor agonists that are more selective than benzodiazepines for the gamma-aminobutyric acid type A (GABA_{A}) receptors that contain the alpha₁ subunit. Binding to the receptors, nonbenzodiazepines enhance GABA-induced ionic currents through the ligand-gated chloride channels, promoting inhibitory effects such as drowsiness. Recently, nonbenzodiazepine drugs have been developed and approved for clinical prescription for the treatment of anxiety. These drugs can produce effective relief of anxiety/panic with little to no sedation, anterograde amnesia, or anticonvulsant effects, and thus may be preferable in some examples to other anti-anxiety drugs.

Nonbenzodiazepines are effective, but are known to have habit-forming potential as well as a number of undesirable side effects. One well-known side effect concerns parasomnias, where users participate in a variety of activities included but not limited to preparing meals, driving, and the like, with little to no recollection of the particular activity(s) performed. These side effects can lead to serious accidents and injuries, including death in some cases.

Combining nonbenzodiazepines with other substances can potentiate such side-effects. One particularly relevant example is the combination of nonbenzodiazepines with alcohol. Alcohol can further increase central nervous system effects of nonbenzodiazepines such as drowsiness, dizziness, and trouble concentrating, and can exacerbate impairments in thinking, judgement, memory and/or reflexes. Despite these known risks, patients exhibit high rates of non-adherence with medical recommendations to avoid alcohol consumption when taking nonbenzodiazepine medications. Such risks are particularly relevant among the elderly population, for whom sleep disorders are prominent and for whom the combination of nonbenzodiazepines with alcohol can lead to increased risks of falls and other injuries. For example, the prevalence of insomnia increases with age, with as much as 50% of elderly people reporting increases in time to initiate sleep, disruption of sleep maintenance, and an overall dissatisfaction with sleep quality. Given the high prevalence of insomnia in the elderly, it is not surprising that the use of hypnotics increases with age, and that such use can contribute to undesirable side effects, the exacerbation of which can be caused by co-consumption of alcoholic beverages.

Thus, the current standard of care for preventing the combining of alcohol with nonbenzodiazepine medications, which is essentially limited to patient counseling, is ineffective. Accordingly, an improved solution is needed for preventing or substantially reducing the combining of alcohol and nonbenzodiazepine medication, to enhance the safety of use of nonbenzodiazepine medications.

Methods of Treatment

Disclosed herein is a method of reducing the chances of a subject experiencing an adverse medically-related event stemming from concomitant ingestion of alcohol whilst taking an NBBRA medication. The disclosed method involves combining an ALDI (e.g., disulfiram [1-diethylthiocarbamoyldisulfanyl-N,N-diethylmethanethioamide]) into a combination drug product, such as a ‘poly-pill’, with an NBBRA (e.g., prescribed NBBRA), for example with one or more of zolpidem, zaleplon, pagoclone, gedocarnil, indiplon, eszopiclone, zopiclone, ocinaplon, and the like, and administering the combination to a subject, such as a subject for whom the NBBRA has been prescribed. Administration of such a combination enables delivery of the NBBRA(s) in a manner that prevents co-consumption of alcohol, thereby in turn reducing a risk of the subject experiencing a medically-related adverse event stemming from the ingestion of alcohol while taking the NBBRA medication.

Existing pharmacotherapies for treating alcoholism include administration of agents that inhibit the enzyme aldehyde dehydrogenase (ALDH), which is involved in the removal of acetaldehyde, a toxic metabolite of alcohol. Although multiple forms of ALDH exist, ALDH-I and ALDH-II are the major enzymes responsible for the oxidation of acetaldehyde. While not being bound by theory, ALDH-I has a higher affinity for acetaldehyde than ALDH-II, and is believed to be the primary enzyme involved in alcohol detoxification. The combination, such as in a poly-pill, of the ALDI with a prescribed NBBRA will prevent the co-consumption of alcohol with prescribed NBBRA medications, because disulfiram and other ALDIs prevent the metabolism of alcohol. Therefore, once an NBBRA-ALDI combination medication is administered, the NBBRA will induce its typical profile of intended effects (e.g., sedative, anxiolytic, and the like), but any subsequent co-consumption of alcohol will result in a strong noxious physiologic reaction to the alcohol, thereby preventing the subject from consuming alcohol to the point where an adverse medically-related event stemming from the synergistic effects of alcohol and the NBBRA may occur.

The disclosed method includes providing and/or administering to a subject a pharmaceutical preparation (e.g., combination drug product) that includes an effective amount of one or more NBBRAs and an effective amount of one or more ALDIs. In this way, a risk that a subject will experience an adverse medically-related event stemming from concurrent ingestion of alcohol while taking an NBBRA may be reduced. Examples of ALDIs include, e.g., disulfiram, calcium carbimide, coprine, cyanamide, 1-aminocyclopropanol, daidzin, cephalosporins, antidiabetic sulfonyl ureas, metronidazole, ampal, benomyl, citral and active isomers thereof, chloral hydrate, chlorpropamide analogs NPI-1 and API-1, CVT-10216, DEAB, gossypol, kynurenine tryptophan metabolites, molinate, nitroglycerin, pargyline and any active metabolites or analogs exhibiting aldehyde dehydrogenase inhibiting activity.

Patients who consume such inhibitors of ALDH experience mild to severe discomfort if they ingest alcohol. Disulfiram, the best known ALDI and sold under the tradenames Cronetal™, Abstenil™, Stopetyl™, Contrain™, Antadix™, Anietanol™, Exhoran™, Antabuse™, Etabuse™, Abstinyl™, Thiuranide™, Esperal™, Tetradine™, Noxal™, Tetraeti™, is a potent irreversible inhibitor of ALDH-II that slightly inhibits ALDH-I. Ingestion of alcohol while taking disulfiram and other aldehyde dehydrogenase inhibitors results in the accumulation of aldehydes, which causes tachycardia, flushing, diaphoresis, dyspnea, nausea and vomiting (also known collectively as the disulfiram or disulfiram-ethanol reaction). Disulfiram consumption produces sensitivity to alcohol which results in a highly unpleasant reaction when the subject ingests even small amounts of alcohol. Thus, in specific embodiments, the dehydrogenase inhibitor comprises, consists essentially of, or consists of disulfiram.

In specific embodiments, the method includes the administration of one or more of disulfiram, calcium carbimide, coprine, cyanamide, 1-aminocyclopropanol, daidzin, cephalosporins, antidiabetic sulfonyl ureas, metronidazole, ampal, benomyl, citral and active isomers thereof, chloral hydrate, chlorpropamide analogs NPI-1 and API-1, CVT-10216, DEAB, gossypol, kynurenine tryptophan metabolites, molinate, nitroglycerin, pargyline, and/or any active metabolites or analogs exhibiting aldehyde dehydrogenase inhibiting activity. In specific embodiments, the method includes the administration of one or more of disulfiram and/or active metabolites thereof, such as S-methyl N,N-diethyldithiocarbamate, S-methyl N,N-diethyldithiocarbamate sulfoxide, and S-methyl N,N-diethylthiocarbamate sulfoxide.

As disclosed herein, the method includes the administration of an NBBRA medication, such as one or more prescription NBBRAs. NBBRA medications within the present disclosure include, but are not limited to, zolpidem, zaleplon, pagoclone, gedocarnil, indiplon, eszopiclone, zopiclone, ocinaplon, mixtures of any of the foregoing, and/or salts of any of the foregoing. Thus, in embodiments, a subject is administered a therapeutically effective amount of one or more of zolpidem, zaleplon, pagoclone, gedocarnil, indiplon, eszopiclone, zopiclone, ocinaplon, alpidem, saripidem, necopidem, suproclone, pazinaclone, suriclone, divaplon, fasiplon, lorediplon, taniplon, panadiplon, abecarnil, ZK-93423, gedocarnil, SL-651,498, CGS-20625, Y-23684, CGS-9896, TPA-023, CL-218,872, NS-2664, TP-003, NS-2710, TP-13, SX-3228, L-838,417, NS-2710, ELB-139, RWJ-51204, GBLD-345, pipequaline, SB-205,384, SX-3228, salts thereof, and/or combinations thereof. In specific embodiments, the one or more NBBRAs comprises, consists essentially of, or consists of zolpidem, eszopiclone, or zaleplon.

In embodiments, the subject is prescribed one or more NBBRA medications for the management of a sleep disorder (e.g., insomnia, restless leg syndrome, and the like). Additionally or alternatively, the subject is prescribed one or more NBBRA medications for the management of an anxiety disorder. Additionally or alternatively, the subject is prescribed one or more NBBRA medications for panic disorder. Other reasons why a particular subject may be prescribed an NBBRA medication are within the scope of this disclosure.

Therapeutic Formulations

Aspects of the present disclosure further concern a combination medication that includes an ALDI and a nonbenzodiazapine medication (e.g., NBBRAs).

In embodiments, the combination medication includes an effective amount of one or more ALDIs. In embodiments, the one or more ALDIs is selected from one or more of disulfiram, calcium carbimide, coprine, cyanamide, 1-aminocyclopropanol, daidzin, cephalosporins, antidiabetic sulfonyl ureas, metronidazole, ampal, benomyl, citral and active isomers thereof, chloral hydrate, chlorpropamide analogs NPI-1 and API-1, CVT-10216, DEAB, gossypol, kynurenine tryptophan metabolites, molinate, nitroglycerin, pargyline and any active metabolites or analogs exhibiting aldehyde dehydrogenase inhibiting activity. In specific embodiments, the ALDI comprises, consists essentially of, or consists of one or more of disulfiram and/or active metabolites thereof, such as S-methyl N,N-diethyldithiocarbamate, S-methyl N,N-diethyldithiocarbamate sulfoxide, and S-methyl N,N-diethylthiocarbamate sulfoxide. In specific embodiments, the aldehyde dehydrogenase inhibitor comprises, consists essentially of, or consists of disulfiram.

In embodiments, the one or more nonbenzodiazepine medications (e.g., NBBRA(s)) included in the combination medication is selected from zolpidem, zaleplon, pagoclone, gedocarnil, indiplon, eszopiclone, zopiclone, ocinaplon, alpidem, saripidem, necopidem, suproclone, pazinaclone, suriclone, divaplon, fasiplon, lorediplon, taniplon, panadiplon, abecarnil, ZK-93423, gedocarnil, SL-651,498, CGS-20625, Y-23684, CGS-9896, TPA-023, CL-218,872, NS-2664, TP-003, NS-2710, TP-13, SX-3228, L-838,417, NS-2710, ELB-139, RWJ-51204, GBLD-345, pipequaline, SB-205,384, SX-3228, mixtures of any of the foregoing, salts of any of the foregoing, and the like. In specific embodiments, the one or more NBBRAs comprises, consists essentially of, or consists of zolpidem, eszopiclone, or zaleplon.

The method of treatment and pharmaceutical formulations of the present disclosure may further include one or more drugs in addition to the nonbenzodiazepine medication and the ALDI, which additional drug(s) may or may not act synergistically therewith. Thus, in certain embodiments, a combination of two or more NBBRAs may be included in the formulation, in addition to the ALDI (or ALDIs). For example, the dosage form may include NBBRAs having different properties, such as half-life, solubility, potency, mechanism of action, and a combination of any of the foregoing. In yet further embodiments, one or more NBBRAs and one or more ALDIS are included, and a further non-NBBRA drug is also included, in addition to the NBBRA medication. In certain embodiments, such non-NBBRA drugs could preferably provide additional analgesia, as insomnia is often associated with pain (e.g., chronic pain) or muscle spasticity. Accordingly, such non-NBBRA drugs may include but are not limited to baclofen, aspirin; acetaminophen; non-sterioidal antiinflammatory drugs (“NSAIDS”), e.g., ibuprofen, ketoprofen, etc.; N-methyl-D-aspartate (NMDA) receptor antagonists, e.g., a morphinan such as dextromethorphan or dextrorphan, or ketamine; cycooxygenase-II inhibitors (“COX-II inhibitors”); and/or glycine receptor antagonists.

Suitable non-steroidal anti-inflammatory agents include ibuprofen, diclofenac, naproxen, benoxaprofen, flurbiprofen, fenoprofen, flubufen, ketoprofen, indoprofen, piroprofen, carprofen, oxaprozin, pramoprofen, muroprofen, trioxaprofen, suprofen, aminoprofen, tiaprofenic acid, fluprofen, bucloxic acid, indomethacin, sulindac, tolmetin, zomepirac, tiopinac, zido-metacin, acemetacin, fentiazac, clidanac, oxpinac, mefenamic acid, meclofenamic acid, flufenamic acid, niflumic acid, tolfenamic acid, diflurisal, flufenisal, piroxicam, sudoxicam or isoxicam, and the like. Useful dosages of these drugs are well known to those skilled in the art.

N-methyl-D-aspartate (NMDA) receptor antagonists are well known in the art, and encompass, for example, morphinans such as dextromethorphan or dextrorphan, ketamine, d-methadone or pharmaceutically acceptable salts thereof. For purposes of the present disclosure, the term “NMDA antagonist” is also deemed to encompass drugs that block a major intracellular consequence of NMDA-receptor activation, e.g. a ganglioside such as GM₁ or GT_{1b} a phenothiazine such as trifluoperazine or a naphthalenesulfonamide such as N-(6-aminothexyl)-5-chloro-1-naphthalenesulfonamide. These drugs can be used to inhibit the development of tolerance to and/or dependence on addictive drugs, and/or to treat pain.

The treatment of chronic pain via the use of glycine receptor antagonists and the identification of such drugs is described in U.S. Pat. No. 5,514,680. COX-2 inhibitors have been reported in the art and many chemical structures are known to produce inhibition of cyclooxygenase-2. COX-2 inhibitors are described, for example, in U.S. Pat. Nos. 5,616,601; 5,604,260; 5,593,994; 5,550,142; 5,536,752; 5,521,213; 5,475,995; 5,639,780; 5,604,253; 5,552,422; 5,510,368; 5,436,265; 5,409,944; and 5,130,311. Certain preferred COX-2 inhibitors include celecoxib (SC-58635), DUP-697, flosulide (CGP-28238), meloxicam, 6-methoxy-2 naphthylacetic acid (6-MNA), MK-966, nabumetone (prodrug for 6-MNA), nimesulide, NS-398, SC-5766, SC-58215, T-614; or combinations thereof.

The dosage form of a disclosed pharmaceutical composition will be determined by the mode of administration chosen. For example, in addition to injectable fluids, oral dosage forms may be employed. Oral formulations may be liquid such as syrups, solutions or suspensions (e.g., oral sprays) or solid such as powders, pills, tablets, or capsules. Methods of preparing such dosage forms are known, or will be apparent, to those skilled in the art.

In embodiments the combination medication is an oral dosage. The oral dosage forms of the disclosure comprise a therapeutically effective amount of an nonbenzodiazepine medication, together with an ALDI, in a therapeutically effective amount that provides a negative, “aversive” physical experience when alcohol is taken in conjunction with the oral dosage form.

The combination of the nonbenzodiazepine medication, together with an ALDI can be employed in admixtures with conventional excipients, i.e., pharmaceutically acceptable organic or inorganic carrier substances suitable for oral administration, known to the art. Suitable pharmaceutically acceptable carriers include but are not limited to water, salt solutions, alcohols, gum arabic, vegetable oils, benzyl alcohols, polyethylene glycols, gelate, carbohydrates such as lactose, amylose or starch, magnesium stearate talc, silicic acid, viscous paraffin, perfume oil, fatty acid monoglycerides and diglycerides, pentaerythritol fatty acid esters, hydroxymethylcellulose, polyvinylpyrrolidone, etc. The pharmaceutical preparations can be sterilized and if desired mixed with auxiliary agents, e.g., lubricants, preservatives, stabilizers, wetting agents, emulsifiers, salts for influencing osmotic pressure buffers, coloring, flavoring and/or aromatic substances and the like. They can also be combined where desired with other active agents, for example other sedative and/or anti-anxiety and/or analgesic agents. For oral administration, particularly suitable are tablets, dragees, liquids, drops, capsules, caplets and gelcaps. The compositions intended for oral use may be prepared according to any method known in the art and such compositions may contain one or more agents selected from inert, non-toxic pharmaceutically excipients which are suitable for the manufacture of tablets. Such excipients include, for example an inert diluent such as lactose; granulating and disintegrating agents such as cornstarch; binding agents such as starch; and lubricating agents such as magnesium stearate. In tablet form, the tablets may be uncoated or they may be coated by known techniques for elegance or to delay release of the active ingredients. Formulations for oral use may also be presented as hard gelatin capsules wherein the active ingredient is mixed with an inert diluent.

Aqueous suspensions containing the above-identified combinations typically include one or more excipients suitable as suspending agents, for example pharmaceutically acceptable synthetic gums such as hydroxypropylmethylcellulose or natural gums. Oily suspensions may be formulated by suspending the above-identified combinations in a vegetable oil or mineral oil. The oily suspensions may contain a thickening agent such as beeswax or cetyl alcohol. A syrup, elixir, or the like can be used wherein a sweetened vehicle is employed. Injectable suspensions may also be prepared, in which case appropriate liquid carriers, suspending agents and the like may be employed.

An oral dosage form according to the disclosure may be provided as, for example, granules, spheroids, beads, pellets (hereinafter collectively referred to as “multiparticulates”). An amount of the multiparticulates which is effective to provide the desired dose of NBBRA over time may be placed in a capsule or may be incorporated in any other suitable oral solid form. Alternatively, the oral dosage form may be in the form of a tablet.

Certain embodiments of the pharmaceutical compositions comprising a nonbenzodiazepine medication (e.g., one or more NBBRAs) and an ALDI (e.g., one or more ALDIs) may be formulated in unit dosage form suitable for individual administration of precise dosages. The amount of active ingredient such as a nonbenzodiazepine medication and an ALDI administered will depend on the subject being treated, the severity of the disorder, and the manner of administration, and is known to those skilled in the art. Within these bounds, the formulation to be administered will contain a quantity of the nonbenzodiazepine medication and the ALDI in an amount effective to achieve the desired effect in the subject being treated.

In particular examples, for oral administration the compositions are provided in the form of a tablet or capsule containing from about 25 mg to 500 mg of the ALDI (e.g., disulfiram), particularly about 25 mg, about 50 mg, about 75 mg, about 100 mg, about 200 mg, about 250 mg, or about 500 mg of the active ingredient, in combination with the dose of the selected nonbenzodiazepine medication necessary to provide the desired effect (e.g., desired level of sedation, anxiety reduction, etc.). This range of doses of disulfiram was selected to facilitate achieving a total daily dose of disulfiram between 250 mg and 500 mg and to allow for wide variability in the frequency of the nonbenzodiazepine medication dosing schedules (e.g., the nonbenzodiazepine medication can be taken one time daily, or more than one time in some examples, such as two times or even three times. In other examples, the nonbenzodiazepine medication need not be taken every day). In one exemplary oral dosage regimen, a tablet containing about 100 mg to about 500 mg, for example 250 mg, of disulfiram in combination with 1-10 mg of zolpidem is administered once per day, thereby providing a typical total daily dose of disulfiram between 100 mg and 500 mg, for example 250 mg. Typical dosage forms of the most common NBBRA medications (e.g., zolpidem, eszopiclone, zaleplon) are formulated as about 1-10 mg. Thus, combinations of NBBRA medication with various doses of disulfiram, for example between 25mg and 500mg to allow flexibility with regard to the frequency of NBBRA dosing, are contemplated. Thus, for example, in the patient who occasionally takes a single NBBRA tablet on an as-needed basis or who takes a single long-acting NBBRA tablet (e.g., on a daily basis), that NBBRA medication would be combined with either 250 mg or 500 mg disulfiram. It is also contemplated herein, that in some examples a patient could take an NBBRA medication at a greater frequency (e.g., more than one time per day), and in such an example the NBBRA medication may be combined with disulfiram at a dose that results in a daily dose of disulfiram between 250 mg and 500 mg. Disulfiram suits this need for flexibility in treating a range of patients with sleep disturbances and/or anxiety because it can be administered in a once-daily or a multiple-times daily manner and still be effective for the entire day. Thus, in embodiments, the disulfiram dose is selected which, when the NBBRA medication is taken in the prescribed manner, results in the delivery of a total daily dose of 250-500 mg disulfiram.

In particular examples, for oral administration the compositions are provided in the form of a tablet or capsule containing from about 1.0 mg to about 10 mg (or even higher, such as 15 mg or 20 mg) of the NBBRA medication, particularly about 1.0, about 2.0 mg, about 2.5 mg, 5 mg, about 10 mg, about 15 mg, or about 20 mg of the NBBRA medication (depending on the potency of the particular NBBRA selected), which accounts for the symptomatic adjustment of the dosage to the subject being treated. In one exemplary oral dosage regimen, a tablet containing from about 1 mg to about 10 mg NBBRA medication is administered one (or in some examples more and in some examples less) time(s) a day.

Single or multiple administrations of the composition comprising the NBBRA medication, together with an ALDI, can be carried out with dose levels and pattern being selected by the treating physician. Generally, single doses are administered in the case of an NBBRA used as a sleep aid. In particular examples, the composition is administered once per day (or in some examples more, such as twice per day or three times per day), every other day, twice a week, weekly, monthly, or as desired (e.g., selectively used for sleep on particular occasions). Treatment will typically continue for some period of time (e.g., one week, one month, etc.), and may in rare circumstances even continue indefinitely, i.e., chronically. Repeat courses of treatment are also possible.

Routes of administration useful in the disclosed methods include but are not limited to oral and parenteral routes, such as intravenous (iv), intraperitoneal (ip), rectal, topical, ophthalmic, nasal, and transdermal. Pharmaceutical compositions comprising a combination drug product that includes a nonbenzodiazepine medication and an ALDI can be administered to subjects by a variety of routes. These include oral, nasal (such as intranasal), ocular, buccal, enteral, intravitreal, or other mucosal (such as rectal or vaginal) or topical administration. Alternatively, administration will be by orthotopic, intradermal subcutaneous, intramuscular, parenteral intraperitoneal, or intravenous injection routes. Such pharmaceutical compositions are usually administered as pharmaceutically acceptable compositions that include physiologically acceptable carriers, buffers, or other excipients.

An effective amount of a nonbenzodiazepine medication (e.g., one or more NBBRAs) and an ALDI (e.g., one or more ALDIs) will depend, at least, on the particular method of use, and the manner of administration of the therapeutic composition. A “therapeutically effective amount” of a composition is a quantity of a specified compound sufficient to achieve a desired effect in a subject being treated. Ideally, a therapeutically effective amount of an ALDI is an amount sufficient to cause a subject to forgo alcohol without a substantial side effect in the subject. For example, a therapeutically effective amount of an ALDI may be an amount sufficient to cause a subject to stop consumption of alcohol (or significantly reduce consumption) before an NBBRA-related adverse event can occur, or before such an adverse medically-related event results in a significant effect (e.g., death, parasomnia, amnestic behaviors). Similarly, a therapeutically effective amount of an NBBRA medication is an amount sufficient to cause the desired effect, such as a desired sedative or anti-anxiety effect, without a substantial side effect in the subject.

The specific dose level and frequency of dosage for any particular subject may be varied and will depend upon a variety of factors, including the activity of the specific compound, the metabolic stability and length of action of that compound, the age, body weight, general health, sex, and diet of the subject, mode and time of administration, rate of excretion, drug combination, and severity of the condition of the subject undergoing therapy.

Typically, preparation of a pharmaceutical composition (for example, for use as a medicament or in the manufacture of a medicament) entails preparing a pharmaceutical composition that is essentially free of pyrogens, as well as any other impurities that could be harmful to humans or animals. The nonbenzodiazepine medication (e.g., NBBRA medication) and an ALDI may be included in pharmaceutical compositions (including therapeutic and prophylactic formulations), which are typically combined together with one or more pharmaceutically acceptable vehicles or carriers and, optionally, other therapeutic ingredients.

To formulate the pharmaceutical compositions, the nonbenzodiazepine medication and the ALDI can be combined with various pharmaceutically acceptable additives, as well as a base or vehicle for dispersion of the compound. Desired additives include, but are not limited to, pH control agents, such as arginine, sodium hydroxide, glycine, hydrochloric acid, citric acid, and the like. In addition, local anesthetics (for example, benzyl alcohol), isotonizing agents (for example, sodium chloride, mannitol, sorbitol), adsorption inhibitors (for example, Tween 80), solubility enhancing agents (for example, cyclodextrins and derivatives thereof), stabilizers (for example, serum albumin), and reducing agents (for example, glutathione) can be included. When the composition is a liquid, the tonicity of the formulation, as measured with reference to the tonicity of 0.9% (w/v) physiological saline solution taken as unity, is typically adjusted to a value at which no substantial, irreversible tissue damage will be induced at the site of administration. Generally, the tonicity of the solution is adjusted to a value of about 0.3 to about 3.0, such as about 0.5 to about 2.0, or about 0.8 to about 1.7.

The nonbenzodiazepine medication (e.g., NBBRA medication) and the ALDI can be dispersed in a base or vehicle, which can include a hydrophilic compound having a capacity to disperse the compound, and any desired additives. The base can be selected from a wide range of suitable compounds, including but not limited to, copolymers of polycarboxylic acids or salts thereof, carboxylic anhydrides (for example, maleic anhydride) with other monomers (for example, methyl (meth)acrylate, acrylic acid and the like), hydrophilic vinyl polymers, such as polyvinyl acetate, polyvinyl alcohol, polyvinylpyrrolidone, cellulose derivatives, such as hydroxymethylcellulose, hydroxypropylcellulose and the like, and natural polymers, such as chitosan, collagen, sodium alginate, gelatin, hyaluronic acid, and nontoxic metal salts thereof. Often, a biodegradable polymer is selected as a base or vehicle, for example, polylactic acid, poly(lactic acid-glycolic acid) copolymer, polyhydroxybutyric acid, poly (hydroxybutyric acid-glycolic acid) copolymer and mixtures thereof. Alternatively or additionally, synthetic fatty acid esters such as polyglycerin fatty acid esters, sucrose fatty acid esters and the like can be employed as vehicles. Hydrophilic polymers and other vehicles can be used alone or in combination, and enhanced structural integrity can be imparted to the vehicle by partial crystallization, ionic bonding, cross-linking and the like. The vehicle can be provided in a variety of forms, including fluid or viscous solutions, gels, pastes, powders, and microspheres.

The nonbenzodiazepine medication (e.g., NBBRA medication) and the ALDI can be combined with the base or vehicle according to a variety of methods, and release of the compound can be by diffusion, disintegration of the vehicle, or associated formation of water channels. In some circumstances, the compound is dispersed in microcapsules (microspheres) or nanocapsules (nanospheres) prepared from a suitable polymer, for example, isobutyl 2-cyanoacrylate (see, for example, Michael et al., J. Pharmacy Pharmacol. 43: 1-5, 1991), and dispersed in a biocompatible dispersing medium, which yields sustained delivery and biological activity over a protracted time.

The nonbenzodiazepine medication (e.g., NBBRA medication) and ALDI combination drug products can alternatively contain as pharmaceutically acceptable vehicles substances as required to approximate physiological conditions, such as pH adjusting and buffering agents, tonicity adjusting agents, wetting agents and the like, for example, sodium acetate, sodium lactate, sodium chloride, potassium chloride, calcium chloride, sorbitan monolaurate, and triethanolamine oleate. For solid compositions, conventional nontoxic pharmaceutically acceptable vehicles can be used which include, for example, pharmaceutical grades of mannitol, lactose, starch, magnesium stearate, sodium saccharin, talcum, cellulose, glucose, sucrose, magnesium carbonate, and the like.

Pharmaceutical compositions for administering the nonbenzodiazepine medication (e.g., NBBRA medication) and the ALDI can be also be formulated as a solution, microemulsion, or other ordered structure suitable for high concentration of active ingredients. The vehicle can be a solvent or dispersion medium containing, for example, water, ethanol, polyol (for example, glycerol, propylene glycol, liquid polyethylene glycol, and the like), and suitable mixtures thereof. Proper fluidity for solutions can be maintained, for example, by the use of a coating such as lecithin, by the maintenance of a desired particle size in the case of dispersible formulations, and by the use of surfactants. In many cases, it will be desirable to include isotonic agents, for example, sugars, polyalcohols, such as mannitol and sorbitol, or sodium chloride in the composition. Prolonged absorption of the compound can be brought about by including in the composition an agent which delays absorption, for example, monostearate salts and gelatin.

For prophylactic and therapeutic purposes, the pharmaceutical compositions can be administered to the subject in a single bolus delivery, via continuous delivery (for example, continuous transdermal, mucosal or intravenous delivery) over an extended time period, or in a repeated administration protocol (for example, by an hourly, daily, or weekly, repeated administration protocol). The therapeutically effective dosage of the compound can be provided as repeated doses within a prolonged prophylaxis or treatment regimen that will yield clinically significant results to alleviate one or more symptoms or detectable conditions associated with a targeted disease or condition as set forth herein.

Therapeutic compositions that include a nonbenzodiazepine medication (e.g., NBBRA medication) and an ALDI can be delivered by way of a pump (see Langer, supra; Sefton, CRC Crit. Ref. Biomed. Eng. 14:201, 1987; Buchwald et al, Surgery 88:507, 1980; Saudek et al, N. Engl. J. Med. 321:574, 1989) or by continuous subcutaneous infusions, for example, using a mini-pump. An intravenous bag solution can also be employed. One factor in selecting an appropriate dose is the result obtained, as measured by the methods disclosed here, as are deemed appropriate by the practitioner. Other controlled release systems are discussed in Langer (Science 249: 1527-33, 1990).

In one example, a pump is implanted (for example see U.S. Pat. Nos. 6,436,091; 5,939,380; and 5,993,414). Implantable drug infusion devices are used to provide patients with a constant and long-term dosage or infusion of a therapeutic agent. Such device can be categorized as either active or passive.

Active drug or programmable infusion devices feature a pump or a metering system to deliver the agent into the patient's system. An example of such an active infusion device currently available is the Medtronic SYNCHRO MED™ programmable pump. Passive infusion devices, in contrast, do not feature a pump, but rather rely upon a pressurized drug reservoir to deliver the agent of interest. An example of such a device includes the Medtronic ISOMED™.

In particular examples, therapeutic compositions are administered by sustained-release systems. Suitable examples of sustained-release systems include suitable polymeric materials (such as, semi-permeable polymer matrices in the form of shaped articles, for example films, or mirocapsules), suitable hydrophobic materials (for example as an emulsion in an acceptable oil) or ion exchange resins, and sparingly soluble derivatives (such as, for example, a sparingly soluble salt). Sustained-release compositions can be administered orally, parenterally, intracistemally, intraperitoneally, topically (as by powders, ointments, gels, drops or transdermal patch), or as an oral or nasal spray. Sustained-release matrices include polylactides (U.S. Pat. No. 3,773,919, EP 58,481), copolymers of L-glutamic acid and gamma-ethyl-L-glutamate (Sidman et al, Biopolymers 22:547-556, 1983, poly(2-hydroxyethyl methacrylate)); (Langer et al., J. Biomed. Mater. Res. 15: 167-277, 1981; Langer, Chem. Tech. 12:98-105, 1982), ethylene vinyl acetate (Langer et al., Id.)y, or poly-D-(−)-3-hydroxybutyric acid (EP 133,988).

Polymers can be used for ion-controlled release. Various degradable and nondegradable polymeric matrices for use in controlled drug delivery are known in the art. In specific examples, the nonbenzodiazepine medication (e.g., NBBRA medication) and the ALDI are contained in a time released and/or tamper proof pill and/or capsule. Example or such capsule formulations are well known in the art and include those formulations sold under the tradename OxyContin® and the like. Examples of such time release formulations can be found for example in U.S. Pat. Nos. 5,478,577; 5,681,585, 5,672,360; 5,958,459; 6,103,261; 6,143,332; 5,965,161; 5,958,452, 5,968,551, 5,681,585, 5,811,126, 5,843,480, 5,681,585, 5,811,126, 5,843,480, 5,849,240, 5,866,164, 5,879,705, 5,891,471, 5,914,131, 5,965,163, 5,968,551, 6,103,261, 6,143,322, 6,245,357, 6,261,599, 6,294,195, 6,419,960, 6,696,066, 7,514,100, 7,514,100, 7,829,120, 7,846,476, 7,988,998, 8,071,119, 8,075,872, 8,114,383, 8,114,384, 8,153,149, 8,153,152, 8,153,661, 8,168,217, 8,192,722, 8,231,898, 8,309,060, 8,323,889, 8,354,124, 8,361,499, 8,362,029, 8,372,432, 8,414,919, 8,415,401, 8,420,056, 8,420,120, 8,445,018, 8,486,448, 8,486,449, 8,487,002, 8,529,948, 8,551,520, 8,597,681, 8,609,683, 8,647,667, 8,653,066, 8,658,631, 8,668,929, 8,685,447, 8,691,270, 8,715,721, 8,722,086, 8,728,522, 8,741,885, 8,753,665, 8,765,178, 8,795,723, 8,808,740, 8,808,745, 8,815,289, 8,821,929, 8,834,925, 8,846,072, 8,846,086, 8,858,963, 8,871,265, 8,877,241, 8,894,987, 8,894,988, 8,911,719, 8,920,833, 8,920,834, 8,927,013, 8,927,014, 8,927,025, 8,937,097, 8,945,614, 8,951,555, 8,951,556, 8,956,644, 8,962,019, 8,974,821, 8,980,291, 8,987,291, 8,999,961, 9,023,394, 9,023,401, 9,034,376, 9,040,084, 9,044,402, 9,050,335, 9,056,052, 9,056,107, 9,060,940, 9,060,976, 9,084,816, 9,095,614, 9,095,615, 9,101,661, 9,132,096, 9,149,533, 9,161,917, 9,198,861, 9,198,863, 9,198,867, 9,205,055, 9,205,056, 9,216,176, 9,226,901, 9,226,907, 9,233,073, 9,233,160, 9,278,074, 9,278,083, 9,289,391, 9,308,170, 9,308,171, 9,320,717, 9,387,174, 9,387,177, 9,393,206, 9,393,207, 9,399,022, 9,402,813, 9,427,407, 9,433,582, 9,433,625, 9,439,866, 9,452,163, 9,456,985, 9,468,636, 9,486,412, 9,486,413, 9,486,451, 9,492,389, 9,492,390, 9,492,391, 9,492,392, 9,492,393, 9,498,444, 9,498,456, 9,504,681, 9,517,207, 9,517,236, 9,517,271, 9,526,704, 9,526,724, 9,539,328, 9,545,380, 9,545,448, 9,555,113, 9,572,779, 9,572,803, 9,572,804, 9,572,805, 9,572,885, 9,579,285, 9,579,389, 9,592,204, 9,616,030, 9,616,055, 9,629,807, 9,629,837, 9,636,303, 9,642,809, 9,655,853, 9,655,861, 9,655,893, 9,655,894, 9,655,971, 9,662,326, 9,662,399, 9,669,022, 9,669,023, 9,669,024, 9,675,581, 9,675,610, 9,675,611, 9,682,077, 9,693,961, 9,694,080, 9,707,179, 9,707,180, 9,707,224, 9,713,611, 9,730,885, 9,737,490, 9,744,136, 9,744,151, 9,750,701, 9,750,703, 9,750,736, 9,757,341, 9,757,371, 9,763,886, 9,763,933, 9,770,416, 9,770,417, 9,775,808, 9,775,809, 9,775,810, 9,775,811, 9,775,812, 9,775,837, 9,789,104, and 9,789,105.

The subject matter of the present disclosure is further illustrated by the following non-limiting Examples.

### EXAMPLES

This example describes exemplary methodology (clinical trial) for testing the efficacy of the disclosed NBBRA-ALDI combination drug product.

A clinical trial is instituted to demonstrate that the combination therapy 1) remains effective as with regard to the desired purpose (e.g., sedative, anti-anxiety medication, etc.) and 2) effectively prevents co-administration of alcohol while the patient is using an NBBRA medication (for example, within 12-24 hours following administration of the last dose of the combination medication).

The following represents one of the possible clinical trials that may be instituted to determine efficacy of the combination medication and methods of treatment disclosed herein.

1. Eligibility Assessment and Enrollment
a. Inclusion Criteria
   i. An individual with a relevant condition (e.g., sleep disorder, anxiety disorder) treated with an NBBRA.
   ii. The individual has a prior history of alcohol abuse or is suspected of non-adherence with recommendations to avoid concomitant alcohol consumption while taking an NBBRA medication.
   iii. Age greater than 18 years
   iv. Adults must be able to understand and sign the informed consent document
   v. Patients must have an ECOG performance score of 0-2.
   vi. Patients must have laboratory and physical examination parameters within acceptable limits by standard of practice guidelines.
b. Exclusion Criteria

Comorbid alcohol dependency (active, not in remission), or NBBRA (or benzodiazepine) use disorder (active, not in remission).
c. Patient Registration
   Patients will be registered on the trial by the principal investigator or their designee using a protocol specific registration form after signing the appropriate informed consent or agreeing by assent.

2. Study Implementation

This is a prospective study of the efficacy of the combination of an aldehyde dehydrogenase inhibitor (ALDI), such as disulfiram, and a nonbenzodiazepine benzodiazepine receptor agonist (NBBRA) medication. As both classes of drugs have proven individual efficacy at achieving the desired clinical outcome when administered individually, toxicity studies would be unnecessary. In an example, the study would include 3 groups plus a control: traditional NBBRA, traditional disulfiram and a comparable NBBRA-disulfiram combination. Pharmacokinetics would also be performed, as there may need to be some adjustment (for example lowering) in the dosing of certain NBBRAs because their metabolism may be slowed by the disulfiram. Patients are asked to complete a wellness rating (e.g., rating related to sleep quality, quality of life, level of wakefulness during the day, etc.) daily or multiple times per day and are advised/counseled on the importance of alcohol avoidance when taking the NBBRA medication. They are selected/notified randomly at multiple intervals throughout the trial (1-3×/wk) that they must provide a urine or blood sample, and complete surveys regarding their consumption of alcohol during the trial. The groups are then compared for safety and efficacy (in terms of both sedation (and/or anti-anxiety effects) and prevention of alcohol co-consumption), side effect profile, and the like.

3. Study Evaluation

Patients will undergo the following evaluations which may be performed within 4 weeks of enrollment:

Detailed History and Physical Examination including, vital signs, ECOG status, demographic information and family history.

Laboratory evaluations: CBC with differential; Chem 20 [Sodium (Na), Potassium (K), Chloride (CI), Total C02 (bicarbonate), Creatinine, Glucose, Urea nitrogen (BUN), Albumin, Calcium total, Magnesium total (Mg), Inorganic Phosphorus, Alkaline Phosphatase, ALT/GPT, AST/GOT, Total Bilirubin, Direct Bilirubin, LD, Total Protein, Total CK, Uric Acid]; PT/PTT.; Blood alcohol concentration; Urine drug screen (with quantitative and qualitative benzodiazepine and/or nonbenzodiazepine analyses).

4. Follow-Up Examinations

Patients following evaluations:
a. physical exam to include vital signs and ECOG status;
b. laboratory evaluations: pharmacokinetic analysis of aldehyde dehydrogenase inhibitor and NBBRA medication.
c. Patients are asked to complete a wellness rating daily or multiple times per day.
d. Patients are advised/counseled on the importance of alcohol avoidance when taking the NBBRA medication.
e. Patients are required to provide urine samples and/or other samples needed to determine blood-alcohol levels at random intervals.
f. Patients complete a survey/questionnaire about their recent level of alcohol consumption at multiple points over the course of the study.
g. Patients agree to inform study investigators if they experience a complication associated with NBBRA use at any point over the course of the study.

5. Data Collection

Data prior to and during the course of the patient's participation will be collected in order to monitor patient eligibility, and will include review of medical and family history records, blood work, and urinary studies.
a. Toxicity Criteria: This study will utilize the Common Terminology Criteria for Adverse Events (CTCAE) version 4 for toxicity and adverse event reporting. CTCAE version 4 is available on the World Wide Web at ctep.info.nih.gov. All appropriate treatment areas should have access to a copy of the CTCAE version 4.
b. Statistical Considerations: A primary objective of this study is to determine the efficacy of the combination medication to provide the desired NBBRA effect while reducing the incidence of co-consumption with alcohol.

6. Rationale for Subject Selection

Subjects will be selected for this protocol based on relevant condition (e.g., sleep disorder, anxiety disorder) managed with NBBRAs and alcohol abuse or non-adherence with recommendations to avoid alcohol consumption while taking NBBRA medication.

7. Data Reporting
a. Routine Data Reporting: All details of patient evaluation, management and treatment will be documented in the patient medical record. The following information may be captured on the CRFs: detailed demographic information including family history; and laboratory results).
b. Serious Adverse Event Reporting Requirements: The following events will be reported: all deaths with the exception of those due to progressive disease; all events that are not listed in the consent form and that are possibly, probably, or definitely related to the research; all serious adverse events (SAEs) that are not listed in the consent form, but are possibly, probably, or definitely related to the research (with the exception of death due to progressive disease). An SAE is defined as an untoward medical occurrence that: resulted in death; was life-threatening; required or prolonged hospitalization; caused persistent or significant disability/incapacity; resulted in congenital anomalies or birth defects; or required intervention to prevent permanent impairment or death.
c. Adverse Event Reporting in the Continuing Review Report: The following events will be presented to provide the information necessary to clearly identify risks to participants and to make a risk:benefit determination: all Grade 2 events that are not in the consent form, but are possibly, probably or definitely related to the research; all Grade 3 and 4 events that are possibly, probably or definitely related to the research; all Grade 5 events regardless of attribution; and all Serious Events regardless of attribution.

Although certain embodiments have been illustrated and described herein, it will be appreciated by those of ordinary skill in the art that a wide variety of alternate and/or equivalent embodiments or implementations calculated to achieve the same purposes may be substituted for the embodiments shown and described without departing from the scope. Those with skill in the art will readily appreciate that embodiments may be implemented in a very wide variety of ways. This application is intended to cover any adaptations or variations of the embodiments discussed herein. Therefore, it is manifestly intended that embodiments be limited only by the claims and the equivalents thereof.

What is claimed is:

1. A method of reducing the chances of an alcohol-mediated nonbenzodiazepine benzodiazepine receptor agonist (NBBRA)-related adverse event occurring during NBBRA usage, comprising:
administering a single composition to a subject in need of a NBBRA, the single composition comprising:
an effective amount of one or more NBBRAs for the management of a sleep or anxiety disorder; and
an effective amount of one or more aldehyde dehydrogenase inhibitors (ALDIs) sufficient to prevent alcohol consumption, the one or more ALDIs selected from disulfiram, calcium carbimide, coprine, cyanamide, 1-aminocyclopropanol, daidzin, cephalosporins, antidiabetic sulfonyl ureas, metronidazole, ampal, benomyl, citral and active isomers thereof, chloral hydrate, chlorpropamide analogs, (benzoyloxy)[4-chlorophenyl)sulfonyl]carbamic acid 1,1 dimethyl ester (NPI-1), 4-chloro-N-ethyl-N-[(propylamino)carbonyl]benzenesulfonamid (API-1), 3-(((3-(4-(methylsulfonamido)phenyl)-4-oxo-4H-chromen-7-yl)oxy)methyl) benzoic acid (CVT-10216), N,N-diethylaminobenzaldehyde (DEAB), gossypol, molinate, nitroglycerin, pargyline, or pharmaceutically acceptable salts thereof, enabling provision of the effects of the one or more NBBRAs in a manner which prevents concomitant alcohol consumption, thereby reducing the risk of alcohol-mediated NBBRA-related adverse events during NBBRA usage for management of sleep and/or anxiety disorders.

2. The method of claim 1, wherein the ALDI is disulfiram, or a pharmaceutically acceptable salt thereof.

3. The method of claim 1, wherein the one or more NBBRAs is one or more prescription NBBRAs.

4. The method of claim 1, wherein the one or more NBBRAs comprises one or more of zolpidem, zaleplon, pagoclone, gedocarnil, indiplon, eszopiclone, zopiclone, ocinaplon, alpidem, saripidem, necopidem, suproclone, pazinaclone, suriclone, divaplon, fasiplon, lorediplon, taniplon, panadiplon, abecarnil, ZK-93423, gedocarnil, SL-651,498, CGS-20625, Y-23684, CGS-9896, TPA-023, CL-218,872, NS-2664, TP-003, NS-2710, TP-13, SX-3228, L-838,417, NS-2710, ELB-139, RWJ-51204, GBLD-345, pipequaline, SB-205,384, and SX-3228.

5. The method of claim 4, wherein the one or more NBBRAs is zolpidem, eszopiclone, or zaleplon.

6. The method of claim 5, wherein the one or more NBBRAs consists essentially of zolpidem, eszopiclone, or zaleplon.

7. The method of claim 1, wherein the subject is prescribed one or more NBBRAs for the management of the sleep disorder.

8. The method of claim 1, wherein the subject is prescribed one or more NBBRAs for the management of the anxiety disorder.

9. A method of reducing the chances of an alcohol-mediated nonbenzodiazepine benzodiazepine receptor agonist (NBBRA)-related adverse event occurring during NBBRA usage, comprising:
administering to a subject in need of a NBBRA a single combination medication, comprising:
an effective amount of an NBBRA for the management of a sleep and/or an anxiety disorder selected from zolpidem, zaleplon, pagoclone, gedocarnil, indiplon, eszopiclone, zopiclone, ocinaplon, alpidem, saripidem, necopidem, suproclone, pazinaclone, suriclone, divaplon, fasiplon, lorediplon, taniplon, panadiplon, abecarnil, ZK-93423, gedocarnil, SL-651,498, CGS-20625, Y-23684, CGS-9896, TPA-023, CL-218,872, NS-2664, TP-003, NS-2710, TP-13, SX-3228, L-838,417, NS-2710, ELB-139, RWJ-51204, GBLD-345, pipequaline, SB-205,384, or SX-3228; and
an effective amount of one or more aldehyde dehydrogenase inhibitors (ALDIs) selected from disulfiram, calcium carbimide, coprine, cyanamide, 1-aminocyclopropanol, daidzin, cephalosporins, antidiabetic sulfonyl ureas, metronidazole, ampal, benomyl, citral and active isomers thereof, chloral hydrate, chlorpropamide analogs, (benzoyloxy)[4-chlorophenyl)sulfonyl]carbamic acid 1,1 dimethyl ester (NPI-1), 4-chloro-N-ethyl-N-[(propylamino)carbonyl]benzenesulfonamid (API-1), 3-(((3-(4-(methylsulfonamido)phenyl)-4-oxo-4H-chromen-7-yl)oxy)methyl) benzoic acid (CVT-10216), N,N-diethylaminobenzaldehyde (DEAB), gossypol, molinate, nitroglycerin, pargyline, or pharmaceutically acceptable salts thereof, enabling provision of the effects of the one or more NBBRAs in a manner which prevents concomitant alcohol consumption, thereby reducing the risk of alcohol-mediated NBBRA-related adverse events during NBBRA usage for management of sleep and/or anxiety disorders.

10. The method of claim 9, wherein the ALDI is disulfiram, or a pharmaceutically acceptable salt thereof.

11. The method of claim 10, wherein the ALDI consists essentially of disulfiram, or a pharmaceutically acceptable salt thereof.

12. The method of claim 9, wherein the one or more NBBRAs comprises zolpidem, eszopiclone, or zaleplon.

13. The method of claim 12, wherein the one or more NBBRAs consists essentially of one or more of zolpidem, eszopiclone, or zaleplon.

14. The method of claim 9, wherein the subject is prescribed one or more NBBRAs for the management of the sleep disorder.

15. The method of claim 9, wherein the subject is prescribed one or more NBBRAs for the management of the anxiety disorder.

16. A combination medication, comprising:
an effective amount of one or more nonbenzodiazepine benzodiazepine receptor agonists (NBBRAs), and
an effective amount of one or more aldehyde dehydrogenase inhibitors (ALDIs) and a pharmaceutically acceptable carrier, wherein the one or more ALDIs is selected from one or more of disulfiram, calcium carbimide, coprine, cyanamide, 1-aminocyclopropanol, daidzin, cephalosporins, antidiabetic sulfonyl ureas, metronidazole, ampal, benomyl, citral and active isomers thereof, chloral hydrate, chlorpropamide analogs (benzoyloxy)[4-chlorophenyl)sulfonyl]carbamic acid 1,1-dimethylethyl ester (NPI-1) and 4-chloro-N-ethyl-N-[(propylamino)carbonyl]benzenesulfonamid (API-1), 3-(((3-(4-(methylsulfonamido)phenyl)-4-oxo-4H-chromen-7-yl)oxy)methyl)benzoic acid (CVT-10216), N,N-diethylaminobenzaldehyde (DEAB), gossypol, kynurenine tryptophan metabolites, molinate, nitroglycerin, pargyline, S-methyl N,N-diethyldithiocarbamate, S-methyl N,N-diethyldithiocarbamate sulfoxide, and S-methyl N,N-diethylthiocarbamate sulfoxide, HNO, 1-aminocyclopropanol (ACP), thioampal, 2-mercaptobenzothiazole (MBT), molinate sulfoxide, molinate sulfone, NO3, propiolaldehyde, or pharmaceutically acceptable salts thereof.

17. The combination medication of claim 16, wherein the ALDI is disulfiram, or a pharmaceutically acceptable salt thereof.

18. The combination medication of claim 16, wherein the ALDI consists essentially of disulfiram, or a pharmaceutically acceptable salt thereof.

19. The combination medication of claim 16, wherein the one or more NBBRAs is one or more prescription NBBRAs.

20. The combination medication of claim 16, wherein the one or more NBBRAs is one or more of zolpidem, zaleplon, pagoclone, gedocarnil, indiplon, eszopiclone, zopiclone, ocinaplon, alpidem, saripidem, necopidem, suproclone, pazinaclone, suriclone, divaplon, fasiplon, lorediplon, taniplon, panadiplon, abecarnil, ZK-93423, gedocarnil, SL-651,498, CGS-20625, Y-23684, CGS-9896, TPA-023, CL-218,872, NS-2664, TP-003, NS-2710, TP-13, SX-3228, L-838,417, NS-2710, ELB-139, RWJ-51204, GBLD-345, pipequaline, SB-205,384, and SX-3228.

21. The combination medication of claim 20, wherein the one or more NBBRAs is zolpidem, eszopiclone, or zaleplon.

22. The combination medication of claim 20, wherein the one or more NBBRAs consists essentially of one or more of zolpidem, eszopiclone, or zaleplon.
